(19) United States
(12) Patent Application Publication
(10) Pub. No.: US 2014/0024944 A1
(43) Pub. Date: Jan. 23, 2014

(54) FLOW VELOCITY ESTIMATION AND ULTRASOUND SYSTEMS FOR FLOW VELOCITY ESTIMATION

(71) Applicant: INDUSTRIAL TECHNOLOGY RESEARCH INSTITUTE, Chutung Hsinchu (TW)

(72) Inventors: Yio-Wha SHAU, Taipei City (TW); Kuo-Tung Tiao, Hsinchu County (TW); Guo-Zua Wu, Taichung City (TW); Chih-Yu Chang, Hsinchu City (TW); Yi-Jung Wang, Kaohsiung City (TW); Kun-Ta Wu, Nantou County (TW); Wan-Yi Chen, Miaoli County (TW)

(73) Assignee: Industrial Technology Research Institute, Chutung Hsinchu (TW)

(21) Appl. No.: 13/728,458

(22) Filed: Dec. 27, 2012

Related U.S. Application Data
(60) Provisional application No. 61/672,298, filed on Jul. 17, 2012.

(51) Int. Cl. A61B 8/08; A61B 8/06; A61B 8/00

(52) U.S. Cl. CPC A61B 8/488; A61B 8/4254; A61B 8/06; A61B 8/4444; A61B 8/461
USPC 600/453

(57) ABSTRACT

Systems and methods for measuring flow velocities, including ultrasound systems, are provided. A Doppler angle between a direction of ultrasound signals and an axis of a flow may be estimated to improve the accuracy of the flow velocity estimation that is based on Doppler effects. A sensor may be mounted on or in an ultrasound probe to obtain a reference orientation of the ultrasound probe and an orientation of the ultrasound probe relative to the reference orientation when the ultrasound probe is moved to other positions. The Doppler angle may be estimated based on the orientation of the ultrasound probe.

### CROSS REFERENCE TO RELATED APPLICATIONS

This application is related to, and claims the benefit of priority of, U.S. Provisional Application No. 61/672,298, filed Jul. 17, 2012, titled “Automated Flow Velocity Calibration in Ultrasound System”, the entire contents of which are incorporated herein by reference.

### TECHNICAL FIELD

The technical field generally relates to ultrasound systems, and more particularly, to flow velocity estimation and ultrasound systems for flow velocity estimation.

### BACKGROUND

Ultrasound systems have become widely-used diagnostic tools for various medical applications. Many ultrasound systems, compared to some other diagnostic tools or systems, are generally non-invasive and non-destructive. As an example, an ultrasound system may include a hand-held probe, i.e., a transducer, for approaching or placing directly on and moving over a subject, such as a patient. The ultrasound system may provide visualization of the subject's internal structures, such as tissues, vessels, and/or organs. The ultrasound system generally works by electrically-exciting transducer elements to generate ultrasound signals, which travel into the body, and by receiving the echo signals reflected from tissues, vessels, and/or organs. The reflected echo signals are then processed to produce a visualization of the subject's internal structures.

One of the applications of ultrasound systems is for measuring blood flow velocity, such as the velocity of blood flow in an artery or a jet of blood flow over or near a heart valve. Such information can be particularly useful in cardiovascular studies and other medical areas. Therefore, it may be desirable to have ways to estimate blood flow velocity in ultrasound systems.

### SUMMARY

Consistent with embodiments, there is provided a method for measuring a flow velocity. The method includes transmitting ultrasound signals to a target object, the ultrasound signals being emitted from an ultrasound signal transmitter in an ultrasound device; detecting ultrasound echo signals resulting from the ultrasound signals emitted to the target object, the ultrasound echo signals being reflective of a flow within the target object; detecting, by a sensor, an orientation of the ultrasound device relative to a reference orientation by a sensor, the reference orientation comprising a Doppler angle of about 90 degrees; estimating a Doppler angle between a primary direction of the ultrasound signals and an axis of the flow within the target object based on at least the orientation of the ultrasound device; and estimating the flow velocity of the flow within the target object based on at least an estimated Doppler angle.

Consistent with embodiments, the reference orientation may be determined based on a Doppler image of a projected flow velocity on the primary direction of the ultrasound signals. The projected flow velocity is approximately zero when the ultrasound device is placed at the reference orientation. The sensor may be mounted on or in the ultrasound device for detecting the orientation of the ultrasound device. The sensor may include one of an accelerometer, a gyroscope, a compass, a OPS receiver, and a camera. A summation of the estimated Doppler angle and the orientation of the ultrasound device may be about 90 degrees. The method may further include performing transmission focusing and reception focusing, by using a beam former, based on relative positions between the target object and the ultrasound device. A chip integrated in an ultrasound system may be configured to estimate the flow velocity based on at least an estimated Doppler angle.

Consistent with embodiments, there is also provided an ultrasound system for measuring a flow velocity, including: an ultrasound device operable to transmit ultrasound signals to a target object and to detect ultrasound echo signals from the target object, the ultrasound echo signals being reflective of a flow within the target object; a sensor for detecting an orientation of the ultrasound device relative to a reference orientation, the reference orientation comprising a Doppler angle of about 90 degrees; and a processing device coupled with the ultrasound device for processing the ultrasound signals and the ultrasound echo signals. The processing device is configured to: estimate a Doppler angle between a primary direction of the ultrasound signals and an axis of the flow within the target object based on at least the orientation of the ultrasound device; and estimate the flow velocity of the flow within the target object based on at least an estimated Doppler angle.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

The accompanying drawings, which are incorporated in and constitute part of this specification, and together with the description, illustrate and serve to explain various examples.

FIG. 1 illustrates an exemplary ultrasound system consistent with certain embodiments of the present disclosure.

FIG. 2 illustrates a block diagram of an exemplary ultrasound system for measuring a blood flow velocity, consistent with an embodiment of the present disclosure.

FIG. 3A illustrates an example of a reference orientation of an ultrasound probe, consistent with an embodiment of the present disclosure.

FIG. 3B illustrates an example of an orientation of an ultrasound probe relative to a reference orientation, consistent with an embodiment of the present disclosure.

FIG. 4 illustrates an exemplary flow chart of an exemplary method for estimating a blood flow velocity, consistent with an embodiment of the present disclosure.

FIG. 5A illustrates an exemplary flow chart of an exemplary method for Doppler mode processing, consistent with an embodiment of the present disclosure.

FIG. 5B illustrates an exemplary flow chart of an exemplary method for B-mode processing, consistent with an embodiment of the present disclosure.

### DETAILED DESCRIPTION

The present disclosure generally relates to systems, methods, and apparatuses for estimating flow velocities in ultrasound systems. The disclosed systems, methods, and apparatuses can be used for estimating a velocity of a blood flow or other types of liquid flow within a subject. In ultrasound systems, blood flow velocity may be estimated based on the Doppler effects, such as by calculating the Doppler shift of a blood flow. Estimating the blood flow velocity is based on the Doppler angle, which is an angle or estimated angle between a primary direction of the ultrasound signals and an axis of a blood flow. And the Doppler shift may vary depending on both the blood flow velocity and the Doppler angle.

To estimate a blood flow velocity based on the Doppler shift, the Doppler angle can be acquired independently or can be estimated by using multiple beams or datasets. The later may involve complicated calculation, data processing, and/or additional equipment. In embodiments consistent with the present disclosure, methods and systems for estimating the Doppler angle are provided. By mounting a sensor on or in the ultrasound probe and detecting an orientation of the ultrasound probe relative to a reference orientation of the ultrasound probe, the Doppler angle can be estimated to provide estimation of the blood flow velocity. Depending on the applications and system designs, an accurate or somewhat more accurate estimation of the blood flow velocity may be obtained compared to some of the traditional estimation methods, in some embodiments, the disclosed methods and systems may enable a convenient and rapid estimation of the Doppler angle without requiring complicated data processing.

Reference will now be made in detail to example approaches implemented consistent with the disclosure; the examples are illustrated in the accompanying drawings. Wherever possible, the same reference numbers will be used throughout the drawings to refer to the same or like parts.

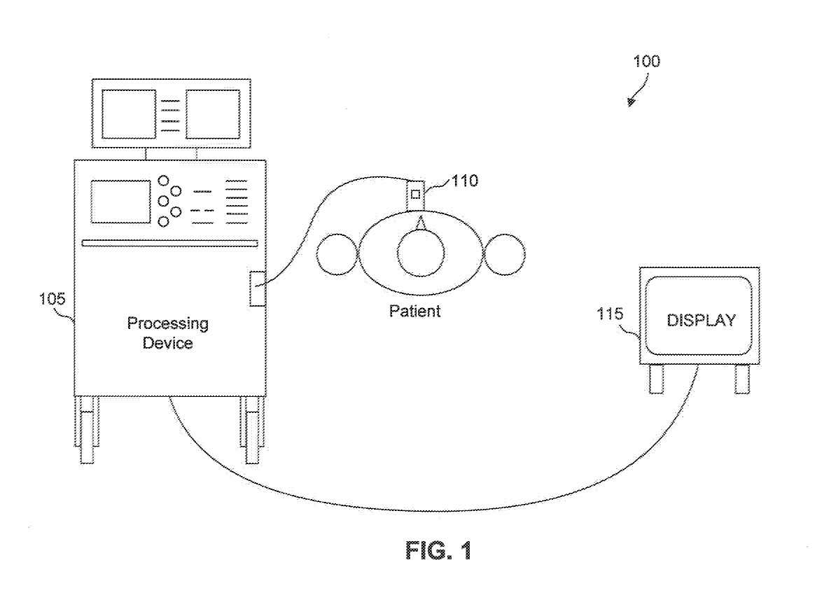

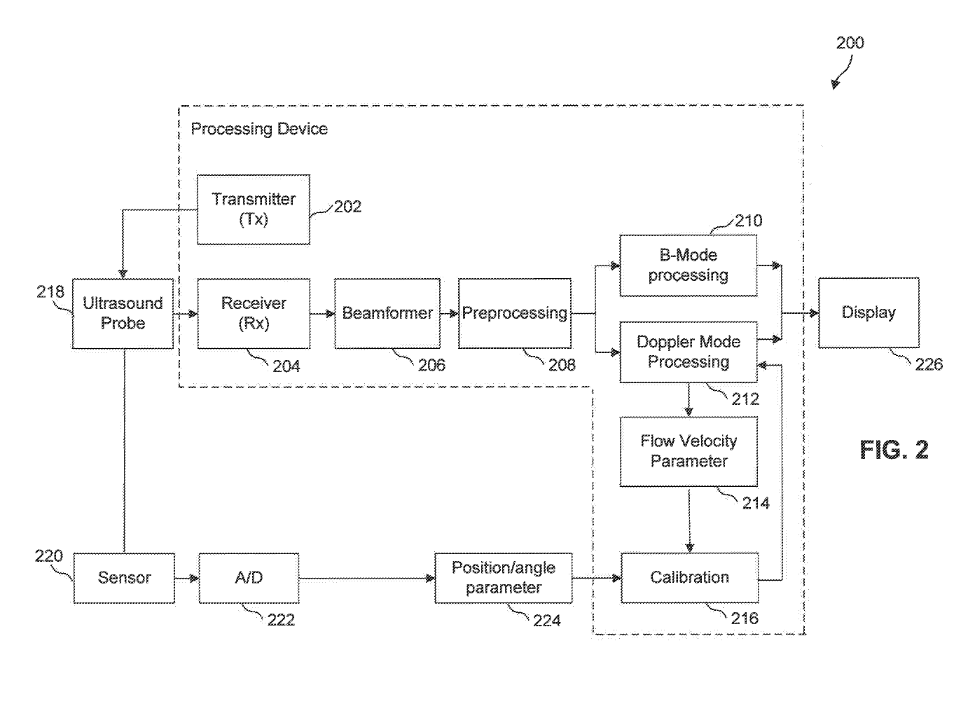

FIG. 1 illustrates an example ultrasound system 100 consistent with certain embodiments of the present disclosure. Referring to FIG. 1, the ultrasound system may include a processing device 105, an ultrasound probe or device 110, and a display 115. Although FIG. 1 illustrates one ultrasound probe 110 and one display 115, the ultrasound system may include one or more ultrasound probes 110 and/or one or more displays 115, which may be based on particular needs, applications, or designs and without departing from the scope of the present disclosure. Also, the various components or devices may be arranged differently. For example, the display may be integrated into the processing device. In a portable system, the processing device 105 and/or the display 115 may be integrated with the ultrasound probe 110. The ultrasound probe may include a sensor, which is illustrated in FIG. 2 as a sensor 220.

The processing device 105 may be a computer or a signal processing device, such as a device that performs various processing and/or control functions related to ultrasound signals. As an example, the processing device 105 may include one or more of the following: a processing module, a memory, one or more signal-amplifiers, and power supplies for the processing module and for the ultrasound probe, a removable storage device (such as floppy disk, optical disk, flash, hard drive, etc.), and a keyboard, which a user of the ultrasound system 100 may use to input data and enter commands for measurements.

The processing module in the processing device may conduct or execute calculations involved in processing the ultrasound data. The processing module can include one or more processing components (alternatively referred to as “processors” or “central processing units” (CPUs)). The processing component can be a central processing unit (CPU), a blade, an application specific integrated circuit (ASK), a field-programmable gate array (FPGA), or other types of processor. The processing module may send signals (such as by supplying currents or applying voltages) to the ultrasound probe 110 for it to emit ultrasound waves, and also receives signals (such as pulses, waveforms, voltages, currents, packets, etc. or any combination of one or more of them) from the ultrasound probe 110 that are generated from the returning echo sound waves. The ultrasound probe may also be referred to as a device in the present disclosure. In one embodiment, the processing module may conduct or execute B-mode processing to generate B-mode images, which is a two-dimensional presentation of the structure(s) within a target object, such as a human's or animal's anatomy. The processing module may also conduct or execute Doppler processing to estimate a flow velocity of the blood flow within the target object. Additionally, the processing module may also send signals that allow the ultrasound images to be displayed on the display 115.

In some embodiments, the processing module may store the processed data and/or images in the memory. As an example, the memory may be a volatile or non-volatile memory, such as, without limitation, magnetic media, optical media, random access memory (RAM), read-only memory (ROM), removable media, or any other suitable memory component. The processing module may also store the processed data and/or images in a disk storage device, such as a hard drive, a floppy drive, an optical disk (CD, DVD, Blu-ray Disk), etc. In some implementations, the processing device 105 may also include or be coupled with a printer to print the ultrasound images. In some implementations, the printer may be connected with the processing device via wireless connections.

The ultrasound probe 110 is coupled with the processing device 105 and may contain an array of piezoelectric crystals to transmit and receive ultrasound signals. As an example, when signals (such as electric currents, voltages, waveforms, etc.) are applied to these crystals, their shapes or forms may vary rapidly based on those signals. The rapid shape changes, or vibrations, of the crystals produce sound waves that travel outwardly. Conversely, when sound or pressure waves hit the crystals, they emit electrical signals. In some examples, the same or different crystals can be used to send and receive sound waves. In some embodiments, the ultrasound probe 110 may also contain a sound absorbing substance or material to reduce or eliminate ultrasound reflections from or echoes caused by the ultrasound probe itself. In one embodiment, the ultrasound probe 110 may also contain an acoustic lens to help focus the emitted sound waves. The processing device 105 may electrically excite the transducer elements to generate ultrasound signals that travel into the patient's body. Echo ultrasound signals reflected from tissues and organs return to the transducer elements and may be converted into electrical signals, which may be amplified and processed by the processing device 105 to produce ultrasound data. In some embodiments, some part of the amplifications and/or processing can be done by or within the ultrasound probe 110.

The ultrasound probe 110 may be moved over or near the surface of the body by an operator of the ultrasound system. In some implementations, a transducer pulse control may be included in the ultrasound system, which may be connected to the processing device 105. The transducer pulse control allows the operator to set and change the frequency and duration of the ultrasound waves. The commands from the transducer pulse control may induce changes of the electric signals that are applied to the piezoelectric crystals in the ultrasound probe 110.

Consistent with an embodiment of the present disclosure, a sensor may be mounted on or in the ultrasound probe 110 to detect an orientation of the ultrasound probe. The sensor may measure either an absolute position of the ultrasound probe 110 or a relative position of the ultrasound probe 110 relative to a prior position or a reference position of the ultrasound probe 110. The sensor can be linear, angular, or multi-axis. For example, the sensor can be an accelerometer, gyroscope, compass, GPS receiver, camera, or any other type of sensor that detects or provides the position or orientation of the ultrasound probe 110. The sensor may feed the position or orientation information of the ultrasound probe 110 to the processing device 105 to estimate a Doppler angle between a primary direction of the ultrasound signals and an axis of a blood flow. The processing device 105 may subsequently estimate a blood flow velocity of an internal organ or tissue of the patient based on the estimated Doppler angle.

The display 115 of the ultrasound system 100 may display various ultrasound data processed by the processing device 105, such as two-dimensional B-mode images, a blood flow velocity in an organ or tissue projected on the direction of the ultrasound beams, the estimated blood flow velocity using Doppler processing, spectral images of the ultrasound echo signals, etc. In some implementations, the display 115 may also display three-dimensional or four-dimensional ultrasound images.

FIG. 2 illustrates a block diagram of an exemplary ultrasound system 200 for measuring a blood flow velocity, consistent with an embodiment of the present disclosure. As shown in FIG. 2, the ultrasound system 200 includes an processing device (202-216), an ultrasound probe with a sensor (218-224), and a display (226).

The processing device includes a transmitter 202 and a receiver 204. The transmitter 202 generates electrical signals to the ultrasound probe to emit ultrasound waves and the receiver 204 receives the electrical pulses from the ultrasound probe that were created from the ultrasound echo signals. The transmitter 202 may adjust the power and/or frequency of the electrical signals to change the power and/or frequency of the ultrasound signals emitted by the ultrasound probe. The receiver 204 may include an amplifier to amplify the received electrical pulses from the ultrasound probe and produce analog or digitized ultrasound data for further processing.

The processing device may include a beam former 206 for performing transmission focusing and reception focusing based on relative positions between the focal points and the transducer elements. Beam forming techniques may be adopted to focus the echo ultrasound signals reflected from different tissue structures in the region of interest. Although not shown in FIG. 2, a transmit beam former may create focused beams of ultrasound by a phased array. In the receive beam former, focusing may be achieved by appropriately delaying echo signals arriving at different transducer elements to align them in a way that creates a pattern of beams pointing in the same direction. These aligned ultrasound echo signals may then be summed coherently. In doing so, processing gain of the received signals can be achieved. The receiving beam forming technique may also be called delay-and-weighting function in the time domain.

The beam former may be implemented in the analog domain or digital domain. In the analog domain, variable analog delay lines may delay the signal from each element or channel, followed by an analog adder. In the digital domain, the signal from each channel is digitized, followed by a memory to implement the phase delay, and a multiplier and adder to sum all delayed channel data. The beam former may be implemented in an application-specific integrated circuit (ASIC), a field-programmable gate array (FPGA), digital signal processor (DSP), or a combination of these components.

The processing unit 208 may be operable to perform signal processing upon reception-focused signals in the beam former. For example, the processing unit 208 may convert analog signal output from the beam former to digital data when an analog beam former is used. Additionally, the processing unit 208 may perform front-end filtering of the received signal to filter out a portion of signal that is not contained within a certain frequency range.

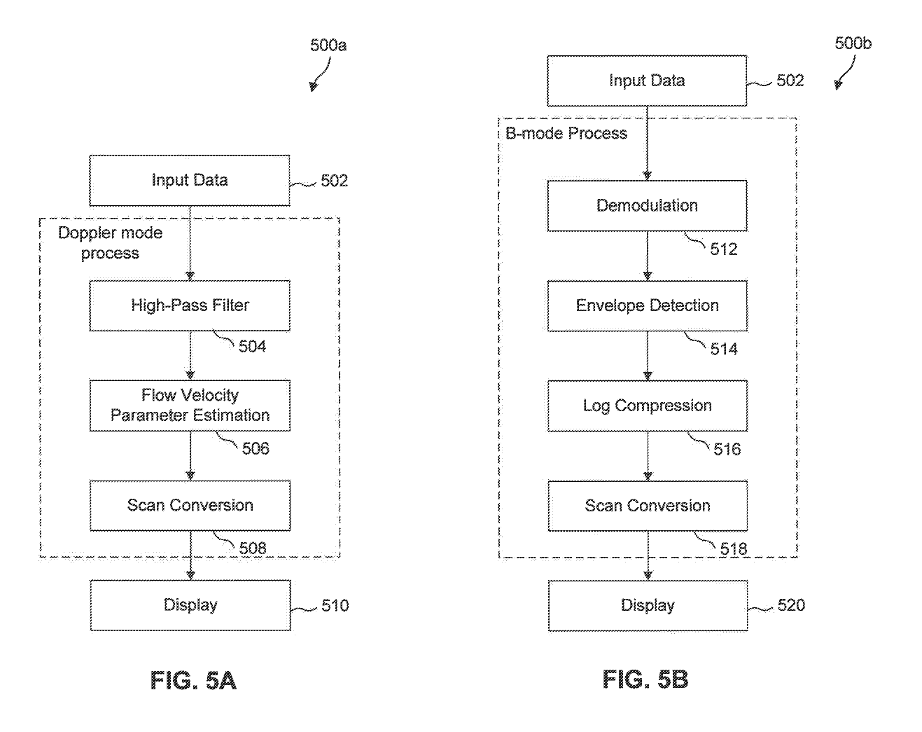

After the preprocessing of the received signals at 208, the output signal from the preprocessing unit may be subjected to a B-mode processing at 210 and a Doppler mode processing at 212. As shown in FIG. 2, the B-mode processing unit 210 and the Doppler mode processing unit 212 are two separate functionalities and thus may be executed in parallel. Results of the B-mode processing and Doppler mode processing may be displayed at the display 226. The detailed function of the B-mode processing and the Doppler mode processing will be described later with reference to FIGS. 5A and 5B.

The Doppler mode processing unit 212 may generate a flow velocity parameter 214 which will be calibrated at 216 based on a position/angle parameter of the ultrasound probe. As shown in FIG. 2, the ultrasound probe 218 is connected with a sensor 220. The sensor 220 may also be mounted on or in the ultrasound probe. The sensor 220 may detect a position or orientation of the ultrasound probe with respect to a reference position or orientation. The position or orientation information detected by the sensor may be converted into digital data at 222.

The digital position or angle parameter of the ultrasound probe 224 may then be input to the calibration unit 216 to calibrate the flow velocity parameter. In other words, the processing device may make use of the position/angle information of the ultrasound probe to further adjust/calibrate the flow velocity parameter at 216 and then output the updated flow velocity parameter back to the Doppler mode processing unit 212. The Doppler mode processing unit 212 may subsequently output the updated flow velocity parameter to display 226 for display.

It should be understood that although the illustrated functionalities of the ultrasound system 200 appear in separate blocks, they may be implemented within an integrated circuit, i.e., a chip. For example, the Doppler mode processing unit 212 and the calibration unit 216 may be integrated into a chip in the processing device for estimating the flow velocity. In some implementations, the B-mode processing unit 210 and the Doppler mode processing 212 may also be integrated into a chip in the processing device. Alternatively, the B-mode processing unit 210, the Doppler mode processing 212, and the calibration unit 216 may be implemented as digital signal processing functions on a FPGA board.

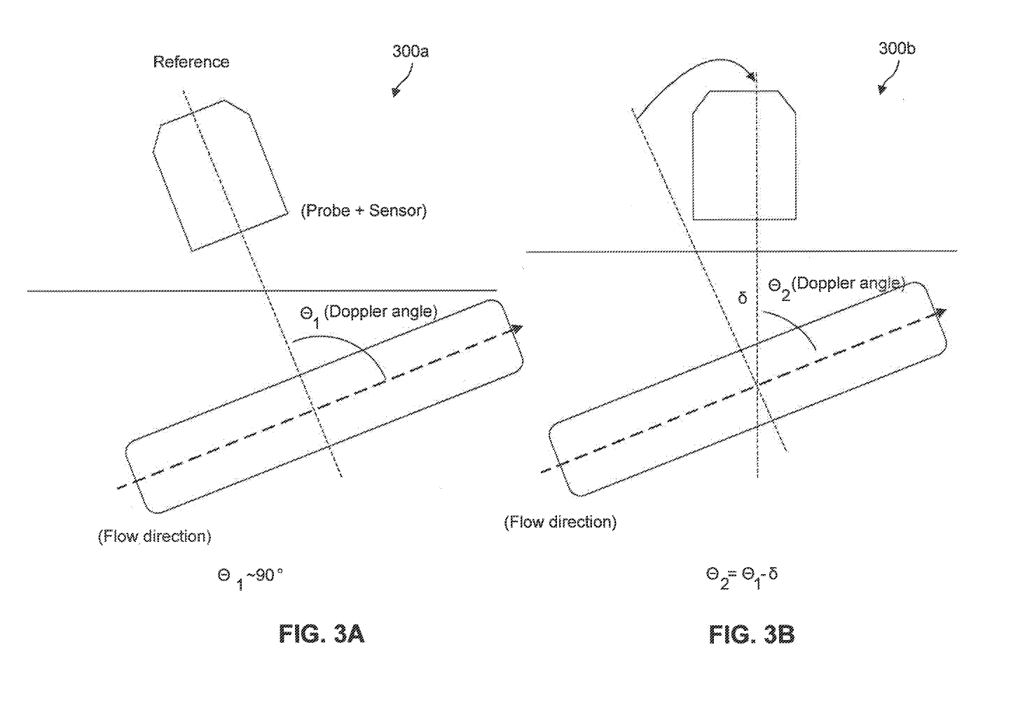

FIG. 3A illustrates an example of a reference orientation of an ultrasound probe 300a, consistent with an embodiment of the present disclosure. As shown in FIG. 3A, the direction of the ultrasound beam and the flow direction are usually different and they form a Doppler angle θ₁. The Doppler angle refers to the angle formed between the primary direction of the ultrasound beam and the flow direction. The Doppler angle affects the projected blood flow velocity on the direction of the ultrasound beam. As mentioned previously, the projected blood flow velocity may be displayed in the display such that an operator operating the ultrasound system may observe it and adjust the position or orientation of the ultrasound probe.

The projected blood flow velocity may be determined by the Doppler mode processing unit 212 (shown in FIG. 2) and be displayed as a Doppler image. For example, the Doppler mode processing unit 212 may calculate the Doppler shift which is the frequency shift between the frequency of the ultrasound echo signals and the ultrasound signals transmitted by the ultrasound probe, and derive the projected blood flow velocity on the direction of the primary ultrasound beam. As the Doppler angle changes, the Doppler shift is different. As a result, the projected blood flow velocity on the direction of the ultrasound beam varies with the Doppler angle.

In order to determine the reference orientation of the ultrasound probe, the operator may move the ultrasound probe in various directions such that the observed projected blood flow velocity is approximately zero. At such position, the Doppler shift is about zero and the Doppler angle is about 90 degrees. In other words, the primary direction of the ultrasound beams is almost perpendicular to the blood flow direction when the ultrasound probe is placed at the reference position or orientation. When the operator finds a position or orientation of the ultrasound probe where the projected blood flow velocity is approximately zero, the operator may set that position or orientation of the ultrasound probe as a reference orientation of the ultrasound probe. The reference position or orientation will be used later to estimate the Doppler angle when the ultrasound probe is moved to other positions for the particular needs of the patient.

FIG. 3B illustrates an example of an orientation of an ultrasound probe 300b relative to the reference orientation, where the ultrasound probe is moved to another position. When the ultrasound probe is moved to another position, the sensor which is mounted on or in the ultrasound probe detects the updated position or orientation of the ultrasound probe and store the orientation of the ultrasound probe δ, with reference to the reference position or orientation. As shown in FIG. 3B, the orientation of the ultrasound probe δ is measured against the reference position or orientation. To obtain the orientation of the ultrasound probe, the sensor may detect a relative movement of the ultrasound probe with reference to the reference position or orientation. In some implementations, the sensor may also detect an absolute position or orientation of the ultrasound probe and calculate the difference between the updated position or orientation of the ultrasound probe and the reference position or orientation. It should be understood that the sensor may be implemented in different ways to detect the position or orientation of the ultrasound probe relative to the reference position or orientation, without departing from the scope of the present disclosure.

Subsequently, the Doppler angle may be obtained based on the position or orientation of the ultrasound probe relative to the reference position or orientation, assuming that the patient does not move during this period and the blood flow direction stays the same when the ultrasound probe moves from the reference position to the updated position. As shown in FIG. 3B, the summation of the Doppler angle Θ₂ and the orientation of the ultrasound probe δ is about 90 degrees. The Doppler angle can be estimated as (90-δ) degrees when the ultrasound probe is placed at the updated position.

Therefore, it is possible to acquire the Doppler angle at any position or orientation of the ultrasound probe, as the sensor detects the orientation of the ultrasound probe relative to the reference position or orientation of the ultrasound probe. The processing device may then estimate the blood flow velocity based on the Doppler shift and the Doppler angle. For example, the Doppler shift f_{d} may be expressed by the following equation:

$${f}_{d} = {f}_{r} - {f}_{s} = \frac{2 v cos θ}{λ} ,$$

where θ represents the Doppler angle, v represents the blood flow velocity, and λ represents the wavelength of the ultrasound waves. Accordingly, the blood flow velocity v may be estimated by the following equation;

$$v = \frac{{f}_{d} \cdot λ}{2 cos θ} .$$

The estimated blood flow velocity may be then displayed at the display in real-time for the operator to perform diagnosis and analysis.

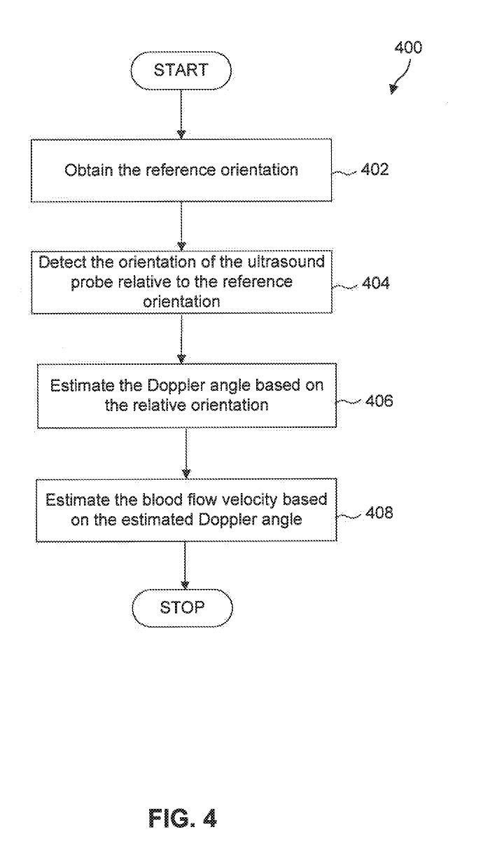

FIG. 4 illustrates an exemplary flow chart of an exemplary method 400 for estimating a blood flow velocity, consistent with an embodiment of the present disclosure. The example method may be executed by the ultrasound system illustrated in FIGS. 1 and 2. As shown in FIG. 4, the reference position or orientation of the ultrasound probe may be obtained at 402. The operator of the ultrasound system may move the ultrasound probe around at this time and observe the projected blood flow velocity on the primary direction of the ultrasound beam on the display. The projected blood flow velocity may be acquired by the Doppler processing unit. When the operator finds a position or orientation of the ultrasound probe at which the projected blood flow velocity is approximately zero, the operator may store this position or orientation as the reference position or orientation of the ultrasound probe. The sensor mounted on or in the ultrasound probe may identify the position or orientation information at the reference position or orientation.

Next, the operator may move the ultrasound probe to a desired position for the diagnosis of the patient. The sensor mounted on or in the ultrasound probe may then detect the orientation of the ultrasound probe at the particular position at 404. The orientation of the ultrasound probe is relative to the reference position or orientation of the ultrasound probe obtained at 402. Note that unless stated otherwise, the orientation of the ultrasound probe is relative to and with reference to the reference position or orientation of the ultrasound probe in the present disclosure. In other words, the orientation of the ultrasound probe is an angle formed between the primary direction of the ultrasound beam when the ultrasound probe is placed at the reference position/orientation and the primary direction of the ultrasound beam when the ultrasound probe is placed at another position.

The sensor may save the orientation of the ultrasound probe in a memory and convert the analog information to digital information through an A/D converter implemented in the sensor. The sensor may then feed the digital information of the orientation of the ultrasound probe to the processing device for Doppler angle estimation.

The processing device may then perform estimation of the Doppler angle based on the orientation of the ultrasound probe at 406. For example, the processing device may estimate the Doppler angle as (90-δ) degrees, where δ represents the orientation of the ultrasound probe. The processing device may also estimate the Doppler angle as (Ω-δ) degrees, where Ω represents a constant angle. Ω may be any number close to 90, such as a number in the range of 80-90 degrees.

The processing device may subsequently estimate the blood flow velocity at 408 based on the estimated Doppler angle. For example, the blood flow velocity v may be estimated as:

$$v = \frac{{f}_{d} \cdot λ}{2 cos θ} .$$

It is also possible to estimate the blood flow velocity using other equations without departing the spirit of the present disclosure. For example, one may estimate the blood flow velocity by taking account of the variance of the estimation error of the Doppler angle or the variance of the measurement of Doppler shift.

In some implementations, multiple measurements of the blood velocity may be conducted and an average of those measurements may be taken as the estimated blood velocity. For example, one may move the ultrasound probe at different positions and repeat steps 404-408, assuming that these positions are still within the interested area with respect to the blood flow velocity. Thus, multiple measurements are performed and multiple sets of blood flow velocity estimation are obtained. An average of these estimations may be used as the estimated blood flow velocity and displayed in the display. Alternatively, a weighted summation can be used as the estimated blood flow velocity, with more weights on the preferred measurements and less weights on the non-preferred measurements. The multiple measurements may mitigate the effects of measurement errors and produce a more reliable estimation of the blood flow velocity. Other methods of combining the multiple measurements are also possible without departing from the scope of the present disclosure.

FIG. 5A illustrates an exemplary flow chart of an exemplary method 500a for Doppler mode processing, consistent with an embodiment of the present disclosure. As shown in FIG. 5A, the input data 502 is first passed to a high-pass filter at 504. Since the ultrasound is scattered from a random distribution of blood cells, the Doppler signal from the blood is distributed at different frequencies. The ultrasound scattered from the pulsating vessel wall may give rise to a low frequency Doppler signal with an amplitude orders of magnitude higher than the signal from the blood. The high-pass filter removes the signal reflected by the vessel wall or other stationary or very slow moving tissues, and thus leaves mainly the signals reflected from blood for further Doppler mode processing.

An infinite impulse response (IIR) filter is usually used as the high-pass filter in Doppler mode processing, such as a 4-pole Butterworth high-pass filter. The cut-off frequency of the high-pass filter may be fixed or be adapted based on the observed spectral image of the received signals. For example, the cut-off frequency may be increased when bright, low-frequency clutter is seen in the spectral image. The low-frequency clutter will be reduced by increasing the cut-off frequency of the high-pass filter.

The Doppler mode processing unit may estimate the flow velocity at 506 based on the signals output from the high-pass filter. The filtered output may be fed to a spectrum analyzer, which typically takes the complex Fast Fourier Transform (FFT) over a moving time window. The Doppler shift may be identified based on the width of the signal spectrum. Each FFT power spectrum may be displayed on the display as a spectral line at a particular time point in the Doppler frequency versus time spectrogram.

The flow velocity parameter can then be estimated based on the measured Doppler shift f_{d}. The flow velocity parameters are the projected blood flow velocity on the primary direction of the ultrasound beam unless it is adjusted/calibrated with the estimated Doppler angle. Thus, the flow velocity parameters may not reflect the true blood flow velocity of the region of interest and may vary when the ultrasound probe points to different directions.

The Doppler mode processing unit may further perform scan conversion at 508 based on the estimated flow velocity parameters. Scan conversion is the process of reformatting ultrasound data for display on a display. Since the coordinate system, in which the ultrasound system usually operates, may not match the display coordinate systems, the scan conversion, which is a coordinate transformation, needs to be performed before displayed on a desired display. Although not shown in FIG. 5A, the Doppler mode processing unit may also perform scan conversion based on the estimated blood velocity which incorporates the Doppler angle, and display the estimated blood flow velocity on the display.

The ultrasound data can be in a Cartesian coordinate (for linear probes) or in polar coordinates (for curvilinear or phase array probes). The scan conversion may transform the coordinates of the ultrasound data to a coordinate that fits the display.

After the scan conversion, the flow velocity parameters may be displayed at 510. A common mode for displaying the blood flow information in real time is the color Doppler velocity mode. It uses color to represent the direction and velocity of the blood flow. As an example, the black line in the center of the color bar may indicate zero velocity. The color in the upper portion of the color bar may represent flow towards the transducer and the color in the lower portion of the color bar may represent blood flow away from the transducer. The shades of the color may indicate the velocity of the blood flow. For example, deep shades indicate low velocities. As the velocity increases, the shade either becomes lighter or changes to another color.

The color representation of the blood flow velocity may be superimposed with B-mode imaging for display. As described previously, the B-mode processing unit may generate B-mode images of the scanned organ or tissue by the ultrasound waves. FIG. 5B illustrates an exemplary flow chart of an exemplary method 500b for B-mode processing. As shown in FIG. 5B, a demodulator demodulates the input data at 512. The demodulator demodulates the ultrasound data into baseband signal components. That is, the demodulation is applied to remove the carrier frequency of the received ultrasound data to extract the complex baseband data, i.e., in-phase (I) and quadrature (Q) components. The baseband data is passed to other function block of B-mode processing for further signal processing.

The baseband data output from the demodulation block is subjected to envelope detection at 514. A low pass filter may be used to eliminate side lobes of the baseband data. The magnitude of the resulting complex signal is then used as detected signal for imaging. The magnitude of the signal is the square root of the sum of the squires of the orthogonal components, i.e., (I²+Q²)^{1/2}. Additional low pass filtering with decimation or interpolation may be carried out on this signal before presenting this signal for further processing.

The output signal from envelop detection is compressed at 516 to fit the dynamic range used for display. The dynamic range of the received signal often exceeds the range that can be displayed by the display. To reduce this dynamic range, a log compressor may be used to achieve the desired dynamic range for display.

Subsequently, the compressed signal is fed to the scan conversion block 518. The scan conversion accepts the processed B-mode vector data, interpolates where necessary, and converts the data into appropriate format for display. Finally, the data output from the scan conversion is displayed at 520. The displayed B-mode image represents a two-dimensional view of the region of interest and provides a convenient tool for clinic diagnosis. The B-mode image may be displayed with the estimated blood flow velocity at the same time to provide more comprehensive information of the patient for the purpose of medical diagnosis and analysis.

The systems and methods described above may be implemented by any hardware, software or a combination of hardware and software having the above described functions. The software code, either in its entirety or a part thereof, may be stored in a computer readable memory.

While several implementations have been provided in the present disclosure, it should be understood that the disclosed systems and methods may be implemented in many other specific forms without departing from the scope of the present disclosure. The present examples are to be considered as illustrative and not restrictive, and the intention is not to be limited to the details given herein. For example, the various elements or components may be combined or integrated in another system or certain features may be omitted, or not implemented. Method steps may be implemented in an order that differs from that presented herein.

Also, techniques, systems, subsystems and methods described and illustrated in the various implementations as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

While the above detailed description has shown, described, and pointed out the fundamental novel features of the disclosure as applied to various implementations, it will be understood that various omissions and substitutions and changes in the form and details of the system illustrated may be made by those skilled in the art, without departing from the intent of the disclosure.

What is claimed is:

1. A method for measuring a flow velocity, the method comprising:
transmitting ultrasound signals to a target object, the ultrasound signals being emitted from an ultrasound signal transmitter in a ultrasound device;
detecting ultrasound echo signals resulting from the ultrasound signals emitted to the target object, the ultrasound echo signals being reflective of a flow within the target object;
detecting, by a sensor, an orientation of the ultrasound device relative to a reference orientation, the reference orientation comprising a Doppler angle of about 90 degrees;
estimating a Doppler angle between a primary direction of the ultrasound signals and an axis of the flow within the target object based on at least the orientation of the ultrasound device; and
estimating the flow velocity of the flow within the target object based on at least an estimated Doppler angle.

2. The method of claim 1, wherein the reference orientation is determined based on a Doppler image of a projected flow velocity on the primary direction of the ultrasound signals.

3. The method of claim 2, wherein the projected flow velocity is approximately zero when the ultrasound device is placed at the reference orientation.

4. The method of claim 1, wherein the sensor is mounted on or in the ultrasound device for detecting the orientation of the ultrasound device.

5. The method of claim 1, wherein a summation of the estimated Doppler angle and the orientation of the ultrasound device is about 90 degrees.

6. The method of claim 1, further comprising performing transmission focusing and reception focusing, by using a beam former, based on relative positions between the target object and the ultrasound device.

7. The method of claim 1, wherein a chip integrated in an ultrasound system is configured to estimate the flow velocity based on at least an estimated Doppler angle.

8. The method of claim 1, wherein the sensor comprises one of an accelerometer, a gyroscope, a compass, a GPS receiver, and a camera.

9. An ultrasound system for measuring a flow velocity, comprising:
an ultrasound device operable to transmit ultrasound signals to a target object and to detect ultrasound echo signals from the target object, the ultrasound echo signals being reflective of a flow within the target object;
a sensor for detecting an orientation of the ultrasound device relative to a reference orientation, the reference orientation comprising a Doppler angle of about 90 degrees; and
a processing device coupled with the ultrasound device for processing the ultrasound signals and the ultrasound echo signals, the processing device configured to:
estimate a Doppler angle between a primary direction of the ultrasound signals and an axis of the flow within the target object based on at least the orientation of the ultrasound device; and
estimate the flow velocity of the flow within the target object based on at least an estimated Doppler angle.

10. The ultrasound system of claim 9, wherein the reference orientation is determined based on a Doppler image of a projected flow velocity on the primary direction of the ultrasound signals.

11. The ultrasound system of claim 10, wherein the projected flow velocity is approximately zero when the ultrasound device is placed at the reference orientation.

12. The ultrasound system of claim 9, wherein the sensor is mounted on or in the ultrasound device for detecting the orientation of the ultrasound device.

13. The ultrasound system of claim 9, wherein a summation of the estimated Doppler angle and the orientation of the ultrasound device is about 90 degrees.

14. The ultrasound system of claim 9, wherein the ultrasound device includes a beam former to perform transmission focusing and reception focusing based on relative positions between the target object and the ultrasound device.

15. The ultrasound system of claim 9, wherein a chip integrated in the ultrasound system is configured to estimate the flow velocity based on at least an estimated Doppler angle.

16. The ultrasound system of claim 9, wherein the sensor comprises one of an accelerometer, a gyroscope, a compass, a GPS receiver, and a camera.
